(19) United States
(12) Patent Application Publication
(10) Pub. No.: US 2004/0224017 A1
(43) Pub. Date: Nov. 11, 2004

(54) Process for preparing sustained release tablets

(76) Inventor: Nirmal Mulye, Princeton, NJ (US)

Correspondence Address: SCULLY SCOTT MURPHY & PRESSER, PC, 400 GARDEN CITY PLAZA, GARDEN CITY, NY, 11530

(21) Appl. No.: 10/800,984

(22) Filed: Mar. 15, 2004

Related U.S. Application Data
(60) Provisional application No. 60/454,803, filed on Mar. 14, 2003.

(51) Int. Cl. A61K 9/22

(52) U.S. Cl. 424/468; 514/58; 514/57

(57) ABSTRACT

The present invention relates to a novel process for preparing sustained release tablets containing medicinal compounds and the products prepared by said process. The present invention is directed to a method of preparing a sustained release pharmaceutical composition having a predetermined drug release profile, but which is deviated therefrom by adding a tableting effective amount of a water insoluble or partially insoluble cellulose, to said pharmaceutical composition comprised of a drug in a therapeutically effective amount, and sustained release carrier present in amounts effective retard the release of the drug from the pharmaceutical composition and said cellulose, the latter being present in an aqueous system, the improvement comprising adding to the composition an effective amount of maltodextrin to retard the increase on the rate of release of the drug from the addition of said cellulose thereto.

### RELATED APPLICATION

The present application is claiming benefit of U.S. Ser. No. 60/454,803 filed on Mar. 14, 2003.

### FIELD OF THE INVENTION

The present invention relates to sustained release pharmaceutical formulations, especially oral sustained release formulations, and the process of preparing said formulations.

### BACKGROUND OF THE INVENTION

Many medical conditions are best treated by administration of a pharmaceutical in such a way as to sustain its action over an extended period of time. Sustained release dosage forms have been used with various types of pharmaceuticals such as anti-hypertensiveness, anti-arrythmics, and the like.

Sustained or timed release compositions containing pharmaceutical medicaments or other active ingredients are designed to contain higher concentrations of an active compound and are prepared in such a manner as to effect sustained or slow release of the compound into the gastrointestinal digestive tract of humans or animals over an extended period of time. Well-absorbed oral sustained or slow release therapeutic drug dosage forms have inherent advantages over conventional, immediate release dosage forms. A less frequent dosing of a medicament, as is required by a sustained release dosage form, increases the resultant patient regime compliance, provides a more sustained drug blood level response, and effects therapeutic action with less ingestion of a drug, thereby mitigating many potential side effects. By providing a slow and steady release of a medicament over time, absorbed drug concentration spikes are mitigated or eliminated by effecting a smoother and more sustained blood level response.

Many physiological factors influence both the gastrointestinal transit time and the release of a drug from a controlled release dosage form. Because such factors can vary from time to time for a particular individual, and can also vary from one individual to another, enzyme or pH dependent sustained release pharmaceutical formulations do not provide a reproducible rate of release of the active pharmaceutical ingredient, and thus do not minimize intra-subject and inter-subject variation in the bioavailability of the active ingredient.

However, whichever method of controlled release is utilized in the pharmaceutical formulation, such as the diffusion of the active ingredient through the coating, erosion of the coating through which the active ingredient passes, diffusion of the active ingredient from a monolithic device, to name a few, the controlled release formulation is required to meet certain criteria. Most importantly, it should result in an uniform and constant dissolution of the active ingredient from the pharmaceutical formulation to be effective for an extended period of time. It is also important that such a formulation be simple to make and that the manufacturing process be reproducible and be useful with a number of different drugs.

In terms of oral administration, tablets have shown to be one of the best methods for administering pharmaceuticals to patients. They have several advantages over capsules. For some drugs, it is recommended that a patient begin taking a smaller dose and gradually over time increase the dose to the desired level to help avoid undesirable side effects. Tablets can be preferable to capsules in this regard because a scored tablet can be broken more easily to form a smaller dose. Further, tablets can be safer to use because they may be less subject to tampering. In addition, tableting processes are generally simpler and less expensive than bead coating and capsule formation.

In common tableting processes, the material which is to be tableted is deposited into a cavity and one or more punch members are then advanced into the cavity and brought into intimate contact with the material to be pressed, whereupon a compression force is applied.

Three basic compression steps are common in most tableting operations, i.e., direct dry compression, wet granulation and dry granulation. Direct compression refers to the compression of a single crystalline compound in the presence of a lubricant and optionally in the presence of additives into a compact tablet form without the use of additional ingredients. Where direct compression is not possible, granulation has been used as a pretreatment wherein materials to be delivered in the tablet are pretreated to form granules that readily lend themselves to tableting. In granulation, the active or intended ingredients are generally admixed with a compression vehicle and/or filler. The compression vehicle or filler must have good compressibility, good flowability and stability under normal ambient conditions as well as being low in cost and satisfactory in both texture and appearance. In addition to compression vehicles, tablet formulations typically include other additives such as diluents, flavor, colors, and disintegrating agents and lubricants, all of which may be added during granulation or thereafter.

Although the wet granulation and the dry granulation methods are the most commonly used, each of them requires several steps in order to prepare a pharmaceutical. For example, the wet granulation process typically includes mixing the components, usually in powdered form; preparing the granulating binder solution; thoroughly mixing the components with the granulating binder solution to form a dough; screening the dough through a sieve; drying, grinding, adding lubricant and compressing the tablets from the resulting mixture. Dry granulation involves the steps of mixing the powder components, compressing the mixture into hard slugs, grinding the slugs into desired particle size, screening, adding excipients, and compressing the mixture into tablets.

In preparing a sustained release formulation, several factors must be taken into consideration. Obviously, the formulation will vary depending upon the identity of the active ingredient. In addition, the interaction of the active ingredient with the other components must also be taken into account. Fundamental to this endeavor is the type and amount of sustained released polymers as well as the process for preparing same.

To prepare sustained release formulations in the form of a solid oral dosage, such as tablets, various hydrophilic polymers have been utilized.

For example, hydroxypropylmethyl cellulose has been used as a polymer for controlled release formulation. For instance, U.S. Pat. No. 4,259,341 to Lowey, U.S. Pat. No. 3,870,190 to Lowey, et al., U.S. Pat. No. 4,226,849 to Schor, and U.S. Pat. No. 4,357,469 to Schor relate to the preparation of tablets having a hydrophilic matrix comprised of hydroxypropylmethyl cellulose alone or mixed with other cellulose derivatives. In addition, U.S. Pat. Nos. 4,369,172 and 4,389,393 to Schor, et al. relate to a sustained release formulation in which the carrier associated therewith contains hydroxypropylmethyl cellulose alone or mixed with methyl cellulose and or sodium carboxy methyl cellulose. Seth in both U.S. Pat. Nos. 4,167,448 and 4,126,672 relates to the use of a pharmaceutical composition containing hydroxypropylmethyl cellulose.

Another polymer that has been used in controlled release formulations is xanthan gum.

U.S. Pat. Nos. 5,292,534 and 5,427,799 to Valentine, et al. disclose a sustained release formulation comprising a pharmaceutical e.g., niacin, with xanthan gum wherein the xanthan gum is present in 20-50 wt % of the formulation.

U.S. Pat. No. 5,415,871 to Pankhania, et al. is directed to a sustained release pharmaceutical formulation comprising xanthan gum, a pharmaceutically active ingredient, for example, ibuprofen or flurbiprofen and other optional excipients. In this formulation, the carrier is at least 50% xanthan gum by weight.

Although comparatively speaking, it is easiest to make sustained or controlled released capsulated pharmaceuticals in oral dosage forms, problems still remain in the making of tableted sustained or controlled release dosages. In some instances, the materials utilized to control the release of the pharmaceutical do not tablet well.

To solve the tableting problem especially, in an extreme case, microcrystalline cellulose, especially silicified microcrystalline cellulose, which is a highly compressible co-processed combination of microcrystalline cellulose with colloidal silicon dioxide, may be used. Commercially, it has superior tableting characteristics and is offered in two grades, one for wet granulations (Prosolv® SMCC.50) and one as a dry binder/diluent (Prosolv® SMCC.90). Unfortunately, when silicified microcrystalline cellulose is used alone in sustained or controlled release tablets, it tends to dramatically speed up the release of a medicament in a pharmaceutical composition.

Although the use of microcrystalline cellulose, especially silicified microcrystalline cellulose, solves one problem by ensuring that proper tableting is formed, it creates another problem for it speeds up the release of the drug from the pharmaceutical composition. An obvious solution to the problem, one would think, would be to increase the amount of controlled release ingredient, for example, hydrophilic polymer when the microcrystalline cellulose, e.g., silicified microcrystalline cellulose, is present. However, the addition of additional hydrophilic polymers in the amounts added to reverse the release effects of the microcrystalline cellulose not only reestablishes the tableting problems, but also makes the drug release too slow. Thus, to date, no one has found the optimal balance between the amount of microcrystalline cellulose to be added and the amount of hydrophilic polymer present, until now.

The present inventor has found a means of preparing a sustained release tablet containing microcrystalline cellulose, including silicified microcrystalline cellulose and sustained release polymers, which does not suffer from tableting problems and which releases the drug present in the formulation at the optimal rate. The present inventor was able to effect this balance by adding an excipient thereto. More specifically, he added maltodextrin thereto. Although maltodextrin is an excipient, he has found it to have drug retarding properties to a small degree. Thus, by adding maltodextrin in effective amounts, the present inventor was able to prepare a controlled release pharmaceutical tablet which does not have the tableting problems and which permits drugs in a sustained release formulation to be released at effective rates.

### SUMMARY OF THE INVENTION

Accordingly, the present invention is directed to a sustained release pharmaceutical for administration of medicinal compounds in a solid unit dosage form, said sustained release formulation comprising:

an active agent; a sustained release carrier or mixture of one or more sustained release carriers, a water insoluble or partially water soluble cellulose, e.g., silicified microcrystalline cellulose, and maltodextrin, wherein the microcrystalline cellulose and the maltodextrin and sustained release carrier or carriers are maintained in an amount effective to permit formation of a solid form of the pharmaceutical composition and to control the release of the active agent.

Such a formulation allows for excellent oral dosage form characteristics, and the maltodextrin is capable of counteracting the increase in the rate associated with the use of the partially water insoluble or fully water insoluble cellulose, such as microcrystalline cellulose, especially silicified microcrystalline.

The present inventor has discovered that oral, unit dosage formulations, especially tablets and pellets, comprising the ingredients described hereinabove and formulated in the manner described herein produce a prolonged action and advantageous delivery system. More specifically, as a result of the methodology used to make the present formulations, a product is obtained which has the desired excellent and regular sustained release pattern. Furthermore, the solid oral dosage forms are prepared in a relatively simple and economical manner.

The present invention is also directed to a method of providing the release of a drug in a sustained release pharmaceutical composition at a predetermined release pattern, which pharmaceutical composition comprises an effective amount of active ingredient and a sustained release polymer present in amounts effective to control the release of the drug, a water insoluble or partially water insoluble cellulose, e.g., silicified microcrystalline cellulose, in an amount effective to enhance formation of the solid oral dosage form of the pharmaceutical composition, said method comprises adding maltodextrin in an amount effective to counteract the increased rate of release from the addition of the cellulose and provide said predetermined release pattern.

The present invention is also directed to a method of administering a sustained release pharmaceutical composition comprising an active ingredient in solid oral dosage form to a patient so that the active ingredient is released at a predetermined rate, said method comprising administering to a patient a therapeutically effective amount of a pharmaceutically active ingredient, a sustained release carrier in an amount effective to retard the release of the drug, the water insoluble or partially water insoluble cellulose in an amount effective to enhance the formation of the oral dosage form of the pharmaceutical composition and maltrodextrin in an amount effective to counteract the increase in rate of release of the drug by the cellulose. It is preferred that the weight ratio of said cellulose to maltodextrin ranges from about 50:1 to about 1:50.

### BRIEF DESCRIPTION OF THE DRAWINGS

FIG. 1 compares the release profile of metronidazole in water at various weight ratios of silicified microcrystalline cellulose and maltodextrin.

FIG. 2 compares a release profile of metformin HCl at various weight ratios of silicified microcrystalline cellulose and metformin.

### DETAILED DESCRIPTION OF THE INVENTION

The present invention is directed to a sustained release pharmaceutical composition for the purpose of administering of medicinal compounds in a controlled form, said sustained release formulation comprising the active agent; a sustained release polymer, a water insoluble or partially soluble cellulose, e.g., microcrystalline cellulose; and maltodextrin, wherein the cellulose and the maltodextrin are maintained in an effective ratio to obtain a controlled sustained release pattern and enhance tableting.

As used herein, the term microcrystalline cellulose includes silicified microcrystalline cellulose.

The term “pharmaceutical”, as employed herein, refers to a medicinally administered composition or compositions as a whole.

As used herein the term “medicinal compound”, “drug”, “active ingredient” and like terms are used interchangeably and as employed herein refers to the active medicament which has a therapeutic effect intended to cure, alleviate, treat or prevent a disease or a symptom or condition suffered by the patient, e.g., hypertension, headaches, pain, high cholesterol levels, and the like. The preferred patient is a mammal, e.g., horse, cow, pig, cat, dog, monkey, mice, rat, human, and the like. The most preferred patient is a human.

The phrase “unit dosage form”, as employed herein, refers to physically discrete units suitable as unitary dosages to human subjects and other mammals, each unit containing a predetermined quantity of active material calculated to produce the desired effect, in association with the other ingredients of the formulation disclosed herein.

The phrase “direct tableting” and like terms, as used herein, signify that the composition can be formed into a tablet using well known tableting apparatus and processes without the need for addition of any additional material to the composition.

As used herein, the term “kp” means kilopounds, a well known unit of force for expressing hardness or crushing strength of pharmaceutical tablets when such hardness is determined.

The percentage of ingredients (a pharmaceutical, polymer, excipients and other ingredients) required in the formulation of the present invention are calculated on a dry weight basis without reference to any water or other components present.

The sustained release formulation of the present invention has an excellent drug profile and is stable with a long shelf life. Moreover, in the sustained release formulation of the present invention, the rate of release of the active agent from the tablet is consistent and uniform among tablets prepared at different times and in different manufacturing batches. The bio-availability characteristics of the tablet prepared in accordance with the procedure herein are substantially uniform among different batches.

The sustained release formulation of the present invention contains an active ingredient. The present formulation is applicable to a wide variety of drugs or active medicaments suitable for use in sustained release formulations.

Representative active ingredients which comprise the pharmaceutical formulation of the present invention include antacids, anti-inflammatory substances, coronary dilators, cerebral dilators, vasodilators, anti-infectives, psychotropics, anti-maniics, stimulants, anti-histamines, laxatives, decongestants, vitamins, gastro-intestinal sedatives, anti-diarrheal preparations, anti-anginal drugs, anti-arrhythmics, anti-hypertensive drugs, vasoconstrictors and drugs for treatment of headaches, including migraines, anti-coagulants and anti-thrombotic drugs, analgesics, anti-pyretics, hypnotics, sedatives, anti-emetics, anti-nauseants, anti-convulsants, neuromuscular drugs, hyper- and hypoglycemic agents, thyroid and anti-thyroid preparations, diuretics, anti-spasmodics, uterine relaxants, mineral and nutritional additives, anti-obesity drugs, anabolic drugs, erythropoietic drugs, anti-asthmatics, bronchodilators, expectorants, cough suppressants, mucolytics and anti-uricemic drugs. Typical active ingredients include gastro-intestinal sedatives such as metoclopramide and propantheline bromide; antacids such as aluminum trisilcate, aluminum hydroxide and cimetidine; anti-inflammatory drugs such as phenylbutazone, indomethacin, naproxen, ibuprofen, fluriprofen, diclofenac, dexamethasone, prednisone and prednisolone; coronary vasodilator drugs such as glyceryl trinitrate, isosorbide dinitrate and pentaerythritol tetranitrate; peripheral and cerebral vasodilators such as solocidilum, vincamine, naftidrofuryl oxalate, co-dergocrine mesylate, cyclandelate, papaverine and nicotinic acid; anti-infective substances such as erythromycin stearate, cephalexin, nalidixic acid, tetracycline hydrochloride, ampicillin, metronidazole, flucloxacillin sodium, hexamine mandelate and hexamine hippurate; neuroleptic drugs such as flurazepam, diazepam, temazepam, amitryptyline, doxepin, lithium carbonate, lithium sulfate, chlorpromazine, thioridazine, trifluoperazine, fluphenazine, piperothiazine, haloperidol, maprotiline hydrochloride, imipramine and desmethylimipramine; central nervous stimulants such as methylphenidate, ephedrine, epinephrine, isoproterenol, amphetamine sulfate and amphetamine hydrochloride; anti-histamic drugs such as diphenhydramine, diphenylpyraline, chlorpheniramine and brompheniramine; laxative drugs such as bisacodyl and magnesium hydroxide; dioctyl sodium sulfosuccinate; nutritional supplements such as ascorbic acid, alpha tocopherol, thiamine and pyridoxine; anti-convulsants such as carbamazepine and 4-methylpyrazole; drugs to treat extrapyramidal movement disorders (such as those associated with parkinsonianism) such as carbidopa and levodopa; anti-spasmodic drugs such as dicyclomine and diphenoxylate; drugs affecting the rhythm of the heart such as verapamil, nifedipine, diltiazem, procainamide, disopyramide, bretylium tosylate, quinidine sulfate and quinidine gluconate; drugs used in the treatment of hypertension such as propranolol hydrochloride, guanethidine monosulphate, methyldopa, oxprenolol hydrochloride, captopril and hydralazine; drugs used in the treatment of migraine such as ergotamine; drugs affecting coagulability of blood such as epsilon aminocaproic acid and protamine sulfate; analgesic drugs such as acetylsalicylic acid, acetaminophen, codeine phosphate, codeine sulfate, carbamazepine, oxycodone, dihydrocodeine tartrate, oxycodeinone, morphine, heroin, nalbuphine, butorphanol tartrate, pentazocine hydrochloride, cyclazacine, pethidine, buprenorphine, and mefenamic acid; anti-epileptic drugs such as phenytoin sodium and sodium valproate; neuromuscular drugs such as dantrolene sodium; substances used in the treatment of diabetes such as tolbutamide, metformin such as metformin salts, e.g., metformin.HCl, disbenase glucagon and insulin; drugs used in the treatment of thyroid gland dysfunction such as triiodothyronine, thyroxine and propylthiouracil; diuretic drugs such as furosemide, chlorthalidone, hydrochlorothiazide, spironolactone and triamterene; the uterine relaxant drug ritodrine; appetite suppressants such as fenfluramine hydrochloride, phentermine and diethylproprion hydrochloride; anti-asthmatic and bronchodilator drugs such as aminophylline, theophylline, salbutamol, orciprenaline sulphate and terbutaline sulphate; expectorant drugs such as guaiphenesin; cough suppressants such as dextromethorphan and noscapine; mucolytic drugs such as carbocisteine; anti-septics such as cetylpyridinium chloride, tyrothricin and chlorhexidine; decongestant drugs such as phenylpropanolamine and pseudoephedrine; hypnotic drugs such as dichloralphenazone and nitrazepam; anti-nauseant drugs such as promethazine theoclate; hemopoietic drugs such as ferrous sulphate, folic acid and calcium gluconate; uricosuric drugs such as sulphinpyrazone, allopurinol and probenecid; hormonal and oral contraceptive drugs such as progesterone and estrogen, and the like. The preferred active ingredients are metformin, carbamazepine, and the like. The drug formulation of the present invention may contain one drug or a combination of two or more drugs.

The active ingredient is present in the pharmaceutical composition in therapeutically effective amounts. It is preferred that the medicament is present in amounts ranging from about 0.5% to about 95% by weight the pharmaceutical composition.

The sustained release carrier useful in the present invention are those sustained release polymers which are used to control the release of medicaments in the pharmaceutical arts. They include sustained release polymers, non-polymer sustained release agents, waxes, and the like. The sustained release polymers include hydrophilic and hydrophobic polymers and waxes, such as a long chain hydrocarbons, long chain alkanoic acid, long chain alkanols and the like. Examples of the sustained release carriers include gums; cellulose ethers; acrylic resins; protein derived materials; digestible long chain C₈-C₅₀ hydrocarbons (containing just hydrogen and carbon), or acids thereof or alcohols thereof or glycerol esters thereof, especially C₁₂-C₄₀ hydrocarbons, such as fatty acids, C₁₂-C₄₀ alcohols, glycerol esters of the fatty acids; mineral and vegetable oils; waxes, especially hydrocarbons having a melting point between 25° C. and 90° C., and polyethylene glycol, and the like. The preferred sustained release carriers are hydrophilic polymers. Preferred hydrophilic polymers include the hydrophilic gums and/or hydrophilic cellulose ethers, polyalkylene oxides, and the like. The hydrophilic gums and cellulose ethers include natural, or partially or totally synthetic, anionic or non-ionic hydrophilic gums, such as, for example, acacia, gum tragacanth, locust bean gum, guar gum, karaya gum, agar, pectin, carrageen, xanthan gum, soluble alginates methyl cellulose, hydroxy propylmethyl cellulose, hydroxypropyl cellulose, hydroxyethyl cellulose, sodium carboxy methyl cellulose, carboxy polymethylene, a combination of two or more hydrophilic gums or cellulose ethers and the like. The preferred hydrophilic polymers are xanthan gum, hydroxypropylmethyl cellulose, or a mixture thereof, as described in U.S. patent application Ser. No. 09/459,300 entitled “Sustained Release Tablet Containing Hydrocolloid and Cellulose Ether”, commonly assigned, the contents of which are incorporated by reference and the like.

Preferred hydrophobic carriers include water insoluble waxes and polymers, such as polyacrylates and polymethacrylates, e.g., Eudragit®, water insoluble cellulose, particularly alkyl celluloses, such as ethyl cellulose, digestible long chain C₈-C₅₀ hydrocarbons, especially C₁₂-C₄₀ alkyl, or fatty acids thereto, fatty alcohols, thereof or glycerol esters thereof, mineral and vegetable oils, and waxes, especially hydrocarbons having a melting point between 25° C. and 90° C.

The preferred hydrophobic polymer is methacylate (Eudragit®) and glyceryl behenate.

The control release carrier is present in effective amounts. It is preferred that the sustained release polymer is present in amounts ranging from about 0.1% to about 50% (w/w) and more preferably from about 1% to about 30% by weight and most preferably from about 2% to about 20% by weight of the composition. If the controlled release polymer is hydrophilic, it is preferred that it is present in an amounts ranging from about 1% to about 50% (w/w) and more preferably from about 2% to about 25% by weight and most preferably from about 3% to about 15% by weight. If the controlled release polymer is hydrophobic, it is preferred that it is present in an amount ranging from about 0.1% to about 50% (w/w) and more preferably from about 1% to about 30% by weight and most preferably from about 2% to about 20% by weight.

The third component of the present formulation is a water insoluble or partially water soluble cellulose, (hereinafter unless indicated to the contrary, designated as “cellulose”). These materials, which are commonly used as excipients, enhance the ability to form tablets. Examples of such materials include microcrystalline cellulose, starch, and the like. The most preferred water insoluble or partially water soluble cellulose is microcrystalline cellulose, especially silicified microcrystalline cellulose. The third component is added in amounts to form a solid oral dosage form, e.g., tablet, capsule, pellets and the like. By forming a solid dosage form, it is meant that it does not disintegrate or fall apart or develop holes or tears under tablet conditions to form a solid dosage form, such as a tablet, capsule, pellet and the like. Moreover, in the case of a tablet, when the various ingredients of the pharmaceutical composition of the present invention are compressed into a tablet, the hardness of the tablet is 5-25 kp.

The amount of the cellulose added depends upon the difficulty in forming a solid dosage form comprised of the drug, the control release polymer and any other ingredients. Preferably, the amount of the third component ranges from about 1% to about 95% by weight of the oral dosage form and more preferably from about 5% to about 65% by weight and most preferably from about 10% to about 50% by weight.

However, in the past, the presence of these excipients has made it difficult to formulate controlled release tablets because they cause disintegration of the tablet when in contact with water. The use of such materials in pharmaceutical compositions can cause the release of the medicament to be more rapid than desired. In some cases, they may even cause failure of the controlled release mechanism and cause dose dumping. Formulations containing such materials may lose hardness on storage at high humidity and create stability problems. Thus, a search was undertaken to find an additional component to add to the formulation to minimize these effects.

For example, the inventor added various components to the formulation, but unfortunately, they tended to make the release profile too slow, and/or did not release the medicament completely in the desired time period so that an effective amount of drug could not be maintained in the bloodstream, thereby adversely effecting the efficacy of the sustained release formulation. For example, the addition of calcium diphosphate, which is not capable of swelling and which has been used in formulations of controlled release matrices, caused the release of the drug to slow down significantly and prevented the complete release of the medication of certain drugs, especially less water soluble drugs.

However, the present inventor has found that the addition of maltodextrin in effective amounts provides the desired release profile. Maltodextrin is a highly hydrophilic polysaccharide which does not swell in the presence of water. Hereforeto, nobody knew maltodextrin also tends to slow down the release of a medicament in a controlled release formulation. The effective amount of maltodextrin added depends upon several factors, including the identity and amount of the drug in the formulation, the identity and the amount of the sustained release carrier and the like. These amounts can be determined by one of ordinary skill in the art without much difficulty. However, the inventor has found that the most important criteria in determining the effective amounts of maltodextrin added depends primarily on the amount of water insoluble cellulose or partially water insoluble cellulose utilized. Thus, the effective amount thereof is added to counteract the accelerated rate of release from the water insoluble or partially water insoluble cellulose, e.g., silicified cellulose. The amount added preferably ranges from a weight ratio of water insoluble or partially water soluble cellulose, e.g., silicified microcrystalline cellulose, to maltodextrin ranging from about 1:50 to about 50:1 and more preferably from about 1:20 to about 20:1 and most preferably from about 1:9 to about 9:1.

In another embodiment, the ratios in the previous paragraphs are preferably the ranges of the total amount of water insoluble or partially water soluble cellulose to the total amount of maltodextrin present in the controlled release formulation of the present invention. Although the oral dosage form may contain water soluble cellulose, such as HPMC, as a sustained release carrier, it is preferred that the total amount of partially water soluble cellulose or insoluble cellulose that is present in the pharmaceutical composition is that amount that enhances the tableting. Moreover, it is preferred that the total amount of maltodextrin present in the pharmaceutical composition is that amount added to counteract the accelerated rate of release attributable to the presence of the partially water soluble or water insoluble cellulose that was added to enhance tableting.

It is preferred that the sum of the water insoluble or partially water insoluble cellulose added and maltodextrin, taken together, ranges from about 5% to about 95% by weight of the oral dosage form and more preferably from about 10% to about 60% by weight with the most preferred range from about 20% to about 50% by weight.

Maltodextrin is an excipient and may be present as a filler in pharmaceutical tablets. However, the maltodextrin used in the present invention is to counteract the accelerated rate of release of the drug attributable to the addition of the water insoluble or partially water insoluble cellulose.

The present inventor has also found another advantage of the present invention. More specifically, the present inventor has found that the water insoluble cellulose or partially water insoluble cellulose in combination with the maltodextrin can be used to fine tune the release profile of the active ingredient from the pharmaceutical composition. This is especially important when the objective is to prepare a sustained release pharmaceutical composition having a desired rate of release. For example, the present invention has found that a small amount of addition of sustained release carrier, e.g., wax, hydrophilic or hydrophobic polymer, has a large effect on the release profile. However, to adjust the release profile of the medicament by just a small amount, the inventor has found that the addition of the water insoluble or partially water insoluble cellulose in combination with maltodextrin slightly modifies the release profile. In other words, the present inventor has found that when the maltodextrin is added in effective amounts to the pharmaceutical composition, the water insoluble or partially water insoluble cellulose in combination with maltodextrin fine tune the release profile. Further, the present inventor has found that maltodextrin and the cellulose derivative can be added to the pharmaceutical composition even if tabelting is not a problem to fine tune the release profile of the medicament from the pharmaceutical composition.

Other additives or adjuvants may additionally be present.

A lubricant may additionally be and is preferably present in the pharmaceutical formulation of the present invention, especially when in the form of a tablet. “Lubricant”, as used herein, refers to a material which can reduce the friction between the die walls and the punch faces which occurs during the compression and ejection of a tablet. The lubricant prevents sticking of the tablet material to the punch faces and the die walls. As used herein, the term “lubricant” includes anti-adherents.

Tablet sticking during its formation and/or ejection may pose serious production problems such as reduced efficiency, irregularly formed tablets and non-uniform distribution of the medicament in the formulation. To avoid this problem, the present invention contemplates utilizing a lubricating effective amount of the lubricant. Preferably, the lubricant is present in amounts ranging from about 0.1% to about 5% by weight and more preferably from about 0.5% to about 2% by weight of the pharmaceutical composition, e.g., tablet. Examples of lubricants include stearate salts, e.g., alkaline earth and transition metal salts, such as calcium, magnesium and zinc stearates; stearic acid, polyethylene oxide; talc; hydrogenated vegetable oil; and vegetable oil derivatives, and the like. In addition, the pharmaceutical composition, e.g., tablet, may contain a combination of more than one type of lubricant. Other lubricants that also can be used include silica, silicones, high molecular weight polyalkylene glycol, monoesters of propylene glycol, and saturated fatty acids containing about 8-22 carbon atoms and preferably 16-20 carbon atoms. The preferred lubricants are the stearate salts, especially magnesium and calcium stearate and stearic acid.

Other excipients, such as plasticizers, for example, diethylphthalate (DEP), dibutyl sebacate, triethyl citrate, triacetin, vegetable and mineral oils, polyethylene glycol, and the like, may optionally be present. Preferably, the plasticizer, when present, is present in the pharmaceutical formations of the present invention in amounts ranging from about 0.1% to about 25%, and more preferably from about 0.1% to about 10% and most preferably form about 1% to about 5% by weight of the carrier.

Other optional ingredients that are also typically used in pharmaceuticals may also be present, such as coloring agents, preservatives (e.g., methyl parabens), artificial sweeteners, flavorants, anti-oxidants, and the like. Artificial sweeteners include, but are not limited to, saccharin sodium, aspartame, dipotassium glycyrrhizinate, stevia, thaumatin and the like. Flavorants include, but are not limited to, lemon, lime, orange and menthol. The colorants include, but are not limited to, various food colors, e.g., FD&C colors, such as FD&C Yellow No. 6, FD&C Red No. 2, FD&C Blue No. 2, food lakes and the like. Examples of anti-oxidants include ascorbic acid, sodium metabisulphite and the like. These optional ingredients, if present, preferably are present in amounts ranging from about 0.1% to about 5% by weight of the tablet and most preferably less than about 3% (w/w) of the tablet.

The formulations of the present invention are preferably uncoated, but may be coated if desired with one of the many readily available coating systems. Nevertheless, it is to be understood that the components described hereinabove, i.e., the drug, drug release polymer, the insoluble or partially insoluble cellulose, maltodextrin and the optional ingredients described hereinabove are present in the core. The coating may be non-functional or functional.

The coating may mask the taste of the pharmaceutical composition of the present invention. Alternatively, coatings may be used to make the unit dosage form of the pharmaceutical composition of the present invention, e.g., tablet, easier to swallow and, in some cases, improve the appearance of the dosage form. The pharmaceutical compositions, e.g., tablet, can be sugar coated; they are sugar coated according to the procedures well known in the art. Alternatively, the unit dosage forms of the pharmaceutical composition of the present invention, e.g., tablets, can be coated with any one of numerous polymeric film coating agents frequently employed by formulation chemists. Representative examples of such film coating agents include hydroxypropyl methylcellulose, carboxymethylcellulose, hydroxypropylcellulose, methyl cellulose, ethyl cellulose, acrylic resins, polyvinyl povidone (PVP), polyvinyl diethylaminoacetate, cellulose acetate phthalate, polyvinyl acetate phthalate, acrylic latex emulsions, ethyl cellulose latex emulsions, and the like.

A procedure for preparing the formulation of the present invention is by the wet granulation process in which all of the components, i.e., medicament, sustained release, carrier, maltodextrin and water insoluble or partially soluble cellulose, e.g., silicified microcrystalline cellulose, any additional excipient and other optional ingredient(s), are mixed with a sufficient amount of a granulating solvent to form a substantially uniform blend in a suitable blender, such as a planetary mixer, Hobart mixer, V blender and the like. The granulating vehicle is one that is inert to the components and has a low boiling point, i.e., preferably less than about 120° C. It is preferably a solvent such as an alcohol containing 1-4 carbon atoms, e.g., isopropyl alcohol or ethanol or water or acetone and the like. An aqueous dispersion can also be utilized, especially if the polymeric sustained release material is a methyl methacrylate copolymer, as described above. In a preferred embodiment, the type of granulating vehicle used is dependent upon the identity of the sustained release polymer. The selection and use of granulating solvent is known to one of ordinary skill in the art. It is preferred that when the sustained release material is a copolymer of methyl methacrylate or ethyl acrylate, the granulating vehicle is an alcohol such as isopropyl alcohol or an aqueous latex dispersion of said copolymer. The ingredients are blended together at effective temperatures. It is preferred that the mixing occurs at room temperature, although slight modifications of temperature therefrom could be utilized. For example, the blending may be effected at temperature ranging from about 10° C. to about 45° C. The ingredients is the formulation are mixed together using techniques will known in the pharmaceutical arts and are intimately intermixed until the mixture is homogenous with respect to the drug.

The substantially uniformly blended mixture may next optionally be milled, e.g., passed through a screen, sieve, etc. to reduce the size of the particles thereof. The screen or sieve, and the like is preferably less than about 140 mesh, and more preferably less than about 100 mesh, and even more preferably, less than about 40 mesh, and most preferably less than about 25 mesh.

Next, the blend is dried. In this step, the solvent is removed from the blend by physical means known to the skilled artisan, e.g., by evaporation or filtration. The resulting granules are again milled, e.g., passed through a screen or sieve to further reduce the size of the particles to the desired size. The lubricant is added, and the granules are mixed to provide a uniform and homogenous blend, and then the resulting mixture is compressed to form a tablet. In a preferred variation, the blend can be simultaneously granulated in the granulation vehicle and dried, such as by using a fluid bed granulation process. Alternatively, the present formulation of the present invention can be prepared by dry formulation by blending the medicament with the lubricant, maltodextrin, water insoluble or partially soluble cellulose and sustained release carrier, and the other optional ingredients. The ingredients are mixed in a typical blender that is normally utilized in the pharmaceutical arts, such as a Hobart mixer, V-blender, a planetary mixer, Twin shell blender and the like. It is preferred that the ingredients are blended together typically at about ambient temperature; no additional heating is necessary, although slight modifications of temperature therefrom could be utilized. For example, the blending be conducted at temperatures ranging from about 10° C. to about 45° C.

The ingredients in the formulation are preferably mixed together such as, e.g., in a large batch, using techniques well known in the pharmaceutical arts and are intimately intermixed until the mixture is homogenous with respect to the drug.

The term “homogenous” with respect to the drug is used to denote that the various components are substantially uniform throughout the invention, i.e., a substantially homogeneous blend is formed.

When the mixture is homogeneous, a unit dosage amount of the mixture is made into a solid dosage form. The formation of the solid dosage form as a tablet is exemplified hereinbelow. However, this is only exemplary for the formation of the pharmaceutical composition of the present invention being made into oral solid dosage forms, which can be effected using techniques known in the art from the homogenous mixture.

In making a tablet, the homogenous mixture is compressed into a tablet form using a tablet machine typically utilized in the pharmaceutical arts. More specifically, the mixture is fed to the die of a tablet press and sufficient pressure is applied to form a solid tablet. Such pressure can vary, and typically ranges from about 1,000 psi to about 6,000 psi and preferably about 2,000 psi force. The solid formulation according to the present invention is compressed to a sufficient hardness to prevent the premature ingress of the aqueous medium into the tablet. Preferably, the formulation is into a tablet form which is of the order of 5-20 Kp and more preferably 8-20 Kp as determined by a Schleuniger hardness test.

In a variation of preparing the drug formulation, all of the above steps are repeated, except that the mixing is initially performed in the absence of a lubricant. When the mixture is homogeneous with respect to the drug, then the lubricant is added and the mixing is continued until the lubricant is substantially evenly dispersed or homogenous in the mixture. Then the mixing is terminated, and the mixture is immediately thereafter compressed into a tablet, as described hereinabove.

When the mixture from either procedure is homogeneous with respect to the drug, a unit dosage form of the mixture is prepared and then compacted, as described hereinabove. This methodology for preparing a tablet containing the pharmaceutical composition of the present invention is exemplary and it is to be understood that the present invention should not be so limited.

After the tablet is formed, the tablet is coated with materials normally used in pharmaceuticals, if desired. If coated, the coating is prepared by techniques known in the art.

As a result of the process described herein, a tablet product is obtained which has the desired hardness and friability typically found for pharmaceutical tablets. The hardness is preferably 5-25 Kp and more preferably 8-20 Kp. In addition, the tablet has an excellent drug release profile. More specifically, it has a predetermined controlled and sustained action release pattern so that the drug is available over a period of up to 36 hours or longer, depending upon the precise tablet size, the identity of the active ingredient, hardness and the particular carrier composition and the needs of the patient. Furthermore, the release profile of each formulation is substantially uniform. Finally, the tablets prepared in accordance with the present invention are hard and dense, have low friability and provide controlled and sustained release over an extended period.

Besides a tablet, the uniformly blended mixture of active ingredient, sustained release carrier, maltodextrin, water insoluble or partially soluble cellulose, e.g., silicified microcrystalline cellulose, can be made into a pellet, capsule, granule, pill or a dragee using conventional techniques known in the art.

Unless indicated to the contrary, all percentages are weight percentages relative to the pharmaceutical composition in solid oral dosage form.

Moreover, the terms “drug” and “medicament” are used interchangeably. Furthermore, the terms “sustained release” and “controlled release” are being used interchangeably.

As used herein, the singular shall refer to the plural and vice versa.

The following non-limiting examples further illustrate the present invention.

### EXAMPLE 1

### Preparation of a Carbidopa/Levodopa Formulation

A controlled-release/sustained-release carbidopa/levodopa tablet containing 53.98 milligrams of carbidopa and 200 milligrams of levodopa is prepared containing the components set forth in Table 1.

Tablets were prepared in accordance with the formulations set forth in Table 1 by passing carbidopa, levodopa, Euragit® RSPO, Prosolv® and Maltodextrin® M180 through a #40 mesh screen wherein these ingredients were mixed in a double cone blender. A suitable mixing time for the ingredients was about 45 minutes. The mixture of carbidopa/levodopa and controlled-release/sustained-release polymer was then mixed with isopropyl alcohol and the wet mass was passed through a #12 mesh screen. The granules were then dried at 60° C. for 2 hours. Afterward, talc and sodium stearyl fumarate were passed through a #40 mesh screen and mixed with the above dried ingredients in a double cone blender, for a suitable mixing time, about 10 minutes.

The above mixture is compressed into white, uncoated, oval, biconvex caplets having a weight of approximately 325 milligrams, a length of about 12.77 millimeters plus or minus 0.02 millimeters, a breadth of about 7.13-7.14 millimeters, a thickness of about 4.61 millimeters plus or minus 0.02 millimeters, and a hardness of about 10-11 Kp.

TABLE 1
Carbidopa/Levodopa Formulation

| | Qty. per | % of |
|---|---|---|
| Ingredient | Tablet (mg) | Formula |
| Carbidopa | 53.98 | 13.11-16.61 |
| Levodopa | 200 | 48.60-61.54 |
| Eudragit RSPO | 13.0 | 3.15-4.0 |
| Prosolv ® 50* | 46.368 | 11.26-14.27 |
| Maltodextrin M180 | 5.152 | 1.25-1.59 |
| Isopropyl alcohol | q.s | 20.02-26.62 |
| Talc | 3.25 | 0.78-1 |
| Sodium stearyl fumerate | 3.25 | 0.78-1 |

*silicified microcrystalline cellulose

### EXAMPLE 2

### Preparation of a Metronidazole Formulation

A controlled-release tablet containing 750 milligrams of metronidazole was prepared. It contained the components, as set forth in Table 2. It was prepared in accordance with the procedure of Example 1. In this formulation the ratio of Prosolv® to Maltodextrin is 3:1.

TABLE 2
Metronidazole Formulation

| Ingredients | Qty per tablet (mg) | % of Formula |
|---|---|---|
| Metronidazole | 750 | 75 |
| Eudragit ® RSPO (5%) | 50 | 5 |
| Prosolv ® 50 | 142.5 | 14.25 |
| Maltodextrin ® | 47.5 | 4.75 |
| Talc (0.5%) | 5 | 0.5 |
| Aerosil (0.5%) | 5 | 0.5 |
| Isopropyl Alcohol | q.s | q.s |
| Totals: | 1000 | 100 |

### EXAMPLE 3

### Preparation of a Metronidazole Formulation

A controlled-release tablet containing 750 milligrams of metronidazole was prepared. It contained the components as set forth in Table 3. It was prepared in accordance with the procedure of Example 1. In this formulation the ratio of Prosolv® to Maltodextrin is 9:1.

TABLE 3
Metronidazole Formulation 2

| Ingredients | Qty per tablet (mg) | % of Formula |
|---|---|---|
| Metronidazole | 750 | 75 |
| Eudragit ® RSPO (5%) | 50 | 5 |
| Prosolv 50 | 171 | 17.1 |
| Maltodextrin M180 | 19 | 1.9 |
| Talc (0.5%) | 5 | 0.5 |
| Aerosil (0.5%) | 5 | 0.5 |
| Isopropyl Alcohol | q.s | q.s |
| Totals: | 1000 | 100 |

### EXAMPLE 4

### Preparation of a Metronidazole Formulation

A controlled-release tablet containing 750 milligrams of metronidazole was prepared. The ingredients used in preparing the tablet is set forth in Table 4. The tablet was prepared in accordance with the procedure of Example 1. The ratio of Prosolv® to Maltodextrin is 18:1.

TABLE 4
Metronidazole Formulation 3

| Ingredients | Qty per tablet (mg) | % of Formula |
|---|---|---|
| Metronidazole | 750 | 75 |
| Eudragit ® RSPO (5%) | 50 | 5 |
| Prosolv ® 50 | 180 | 18 |
| Maltodextrin M180 | 10 | 1 |
| Talc (0.5%) | 5 | 0.5 |
| Aerosil (0.5%) | 5 | 0.5 |
| Isopropyl Alcohol | q.s | q.s |
| Totals: | 1000 | 100 |

### COMPARATIVE EXAMPLE 1

### Preparation of Metronidazole

A controlled release tablet of metronidazole was prepared from the ingredients, set forth below. The tablet is prepared in accordance with the procedure of Example 1.

| Ingredients | Qty per tablet (mg) |
|---|---|
| Metronidazole | 750 |
| Eudragit ® RSPO (5%) | 50 |
| Prosolv ® 50 | 190 |
| Talc (0.5%) | 5 |
| Aerosil (0.5%) | 5 |
| Isopropyl Alcohol | q.s |
| Tablet Weight: | 1000 |

### EXAMPLE 5

### Effects of Maltodextrin on Metronidazole Formulations

The effects of maltodextrin and Prosolv® on the release rate of various metronidazole formulations were tested. Differing ratios of Prosolv® to maltodextrin were tested wherein the percentages of Prosolv tested were 100%, 95%, 90% and 75%. The metronidazole formulations were made in accordance with Examples 2-4 as recited above. The time required to release the metronidazole in water was tested and the results are shown in Table 5.

It is apparent that the ratio of Prosolv® to maltodextrin is critical in affecting the release of an active agent such as metronidazole. Maltodextrin successfully slowed the rate of release of a tablet containing Prosolv® when utilized in an effective amount by as much as 35% thus enabling the pharmaceutical to continue acting over time.

TABLE 5
Release over time of Metronidazole in Water

| | | % | % | % | % |
|---|---|---|---|---|---|
| | | Released | Released | Released | Released |
| | Prosolv ®: | in H₂O at | in H₂O at | in H₂O at | in H₂O at |
| Examples | Maltodextrin | 1 hr | 3 hrs | 5 hrs | 7 hrs |
| Com- | 1:0 (100% | 33.80 | 50.45 | 61.61 | 76.54 |
| parative | Prosolv ®) | | | | |
| Example 1 | | | | | |
| 4 | 19:1 (95% | 34.04 | 58.28 | 68.99 | 78.42 |
| | Prosolv ®) | | | | |
| 3 | 9:1 (90% | 22.57 | 38.85 | 49.77 | 56.85*⁽¹⁾ |
| | Prosolv ®) | | | | |
| 2 | 3:1 (75% | 17.66 | 35.34 | 45.56 | 51.10 |
| | Prosolv ®) | | | | |

*⁽¹⁾At 9 hours the amount of drug release was 62.17% while at 12 hours, the amount of drug released was 70.90%.

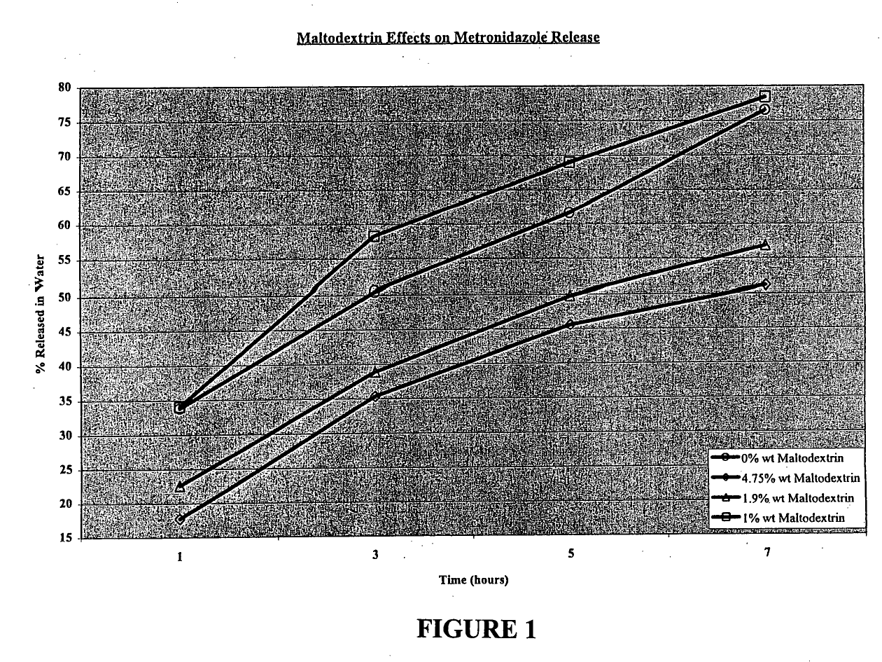

The results are graphically depicted in FIG. 1.

### EXAMPLE 6

### Preparation of a Metformin HCl Formulation

A controlled-release tablet containing metformin HCl was prepared from the components set forth in Table 6. The tablet was prepared by mixing the components in a V-blender for about 1.5 to 2 hours and then compressing the mixture using a tablet press. The ratio of Prosolv® to Maltodextrin is 1:1.

TABLE 6
Metformin HCl Formulation 1

| Ingredients | Qty per tablet (mg) | % of Formula |
|---|---|---|
| Metformin HCl | 500 | 50 |
| Xanthan gum CR (5%) | 50 | 5 |
| Methocel ® E10MCR (15%) | 150 | 15.0 |
| Prosolv ® 50 | 145 | 14.5 |
| Maltodextrin M180 | 145 | 14.5 |
| Aerosil (0.5%) | 5 | 0.5 |
| Magnesium stearate (0.5%) | 5 | 0.5 |
| Total | 1000 | 100 |

### EXAMPLE 7

### Preparation of a Metformin HCl Formulation

A controlled-release tablet containing metformin HCl has been prepared from the ingredients set forth in Table 7. The tablet was prepared in accordance with the procedure of Example 6. The ratio of Prosolv® to Maltodextrin is 9:1.

TABLE 7
Metformin HCl Formulation 2

| Ingredients | Qty per tablet (mg) | % of Formula |
|---|---|---|
| Metformin HCl | 500 | 50 |
| Xanthan gum CR (5%) | 50 | 5 |
| Methocel ® E10MCR (15%) | 150 | 150 |
| Prosolv ® 50 | 261 | 26.1 |
| Maltodextrin M180 | 29 | 2.9 |
| Aerosil (0.5%) | 5 | 0.5 |
| Magnesium stearate (0.5%) | 5 | 0.5 |
| Total | 1000 | 100 |

### COMPARATIVE EXAMPLE 2

### Preparation of a Metformin HCl Formulation

A controlled-release tablet containing metformin HCl has been prepared from the ingredients set forth in Table 8 below. The tablet was prepared in accordance with the procedure of Example 6.

TABLE 8
METFORMIN HCl Formulation

| Ingredients | Qty per tablet (mg) |
|---|---|
| Metformin HCl | 500 |
| Xanthan gum CR (5%) | 50 |
| Methocel ® E10MCR (15%) | 150 |
| Prosolv ® 50 | 290 |
| Aerosil (0.5%) | 5 |
| Magnesium stearate (0.5%) | 5 |
| Tablet Weight | 1000 |

### EXAMPLE 8

### Comparative Study

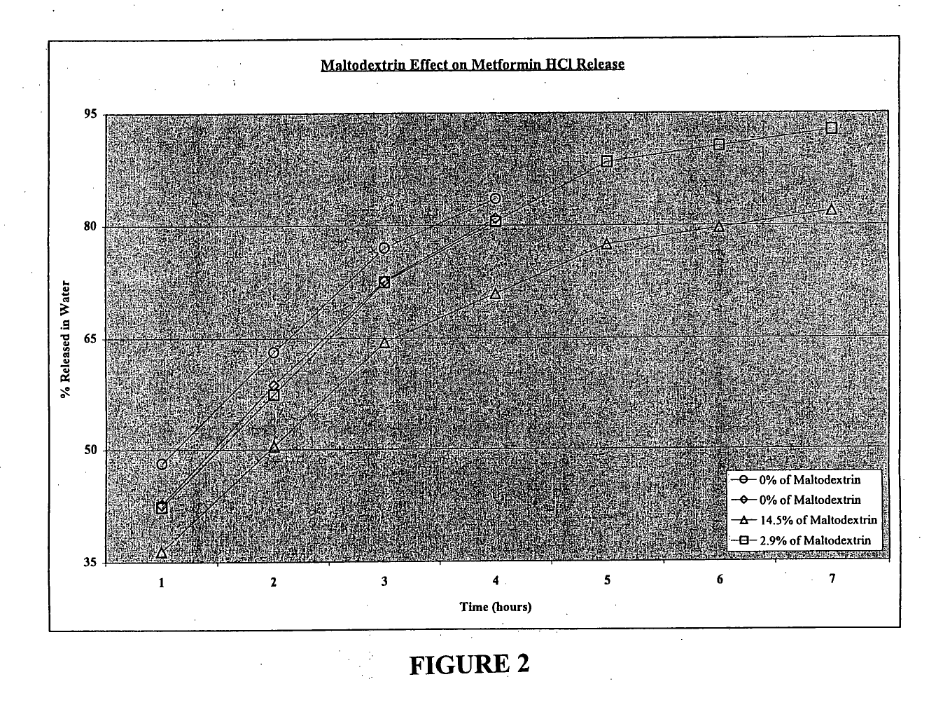

A comparative study was performed of the various metformin HCl tablets prepared in Examples 6 and 7 and Comparative Example 2. Differing ratios of Prosolv® to maltodextrin were tested. The time required to release the drug in water was tested and the results are shown hereinbelow in Table 9 and depicted in FIG. 2.

TABLE 9
Release Profile

| | Prosolv ®, | % released in | % released in | % released in | % released in | % released in | % released in H₂O |
|---|---|---|---|---|---|---|---|
| Example | Maltodextrin Ratio | H₂O in 1 hour | H₂O in 2 hours | H₂O in 3 hours | H₂O in 4 hours | H₂O in 5 hours | in 7 hours |
| Comparative | 100% Prosolv ® | 48.13 | 63.13 | 77.01 | 83.59 | — | — |
| Example 2 | | | | | | | |
| Another | 100% Prosolv ® | 42.66 | 58.69 | 72.66 | 80.95 | — | — |
| Comparative | | | | | | | |
| Example 3 | | | | | | | |
| Example 6 | 1:1 | 36.44 | — | 64.43 | — | 77.45 | 81.87 |
| Example 7 | 9:1 | 42.31 | — | 72.48 | — | 88.53 | 92.73 |

### EXAMPLE 9

### Preparation of Mesalamine Formulation

A controlled release formulation in pellet form was prepared using the following components in the amounts indicated in Table 10.

TABLE 10
MESALAMINE FORMULATION

| INGREDIENTS | QUANTITY PER TABLET (g) |
|---|---|
| Mesalamine | 500 |
| Silicified Microcrystalline Cellulose | 250 |
| Maltodextrin | 100 |
| Surelease ®* | 150 (600) |
| Water | 60 |

*Surelease is ethyl cellulose aqueous dispersion containing 25% w/w solids. 150 (600) refers to 150 g ethyl cellulose, but the total amount of Surelease is 600 g.

The beads were prepared by mixing Mesalamine, silicified microcrystalline cellulose, Maltodextrin in a blender and the mixture of Surelease® and water were added thereto while mixing. The resulting wet mass product was passed through an extruder with 1.25 mm screen to obtain elongated cylinders. The extrudate was then spheronized by a spherionizer to form the solid product. After spheronization, the pellets are dried and placed into hard gelatin capsules.

### COMPARATIVE EXAMPLE 3

A controlled release formulation was prepared in pellet form using the following components in the amounts indicated in the following Table 11.

TABLE 11

| INGREDIENT | QUANTITY PER TABLET (g) |
|---|---|
| Mesalamine | 500 |
| Silicified Microcrystalline Cellulose | 350 |
| Maltodextrin | — |
| Surelease ® | 150 (600) |
| Water | 70 |

The beads were prepared as in Example 9. After spheronization, the pellets are dried and placed into hard gelatin capsules.

### EXAMPLE 10

### Comparative Study

A comparative study was preformed of the mesalamine formulations prepared in Example 9 and Comparative Example 3. The release profile in water is tabulated hereinbelow.

Dissolution Profile

Apparatus: USP I(basket)

Medium: 0.1 N HCl

Speed: 100 rpm

% Release in H₂O

| TIME (Hours) | Comp. Ex. 4 | Comp. Ex. 13 |
|---|---|---|
| 1 | 100 | 76 |
| 2 | | 90 |
| 3 | | 93 |

### EXAMPLE 11

### Mesalamine Formulations

The controlled release pellet was formed in accordance with the procedure in Example 9. The beads obtained after spheronization were dried and were further coated using aqueous dispersion of ethylcellulose and hydroxypropylmethyl cellulose.

### EXAMPLE 12 AND 13 AND COMPARATIVE EXAMPLE 4

### Prepartion of a Clarithromycion Formulation

Three control release tablets containing 500 mg of clarithromycin were prepared. The compositions of each of the formulations are set forth in Table 12.

TABLE 12
Clarithromycin Formulations

| | Formulation I | Formulation | Formulation |
|---|---|---|---|
| | (Comparative | II | III |
| Ingredient | Example 4) | Example 12 | Example 13 |
| Clarithromycin | 500 | 500 | 500 |
| Glyceryl behenate | 25 | 25 | 25 |
| Silicified Microcrystalline | 440 | 330 | 210 |
| Cellulose | | | |
| Maltodextrin | — | 110 | 210 |
| PEG 3350 | 20 | 20 | 20 |
| Magnesium Stearate | 15 | 15 | 15 |
| Ratio: SMCC:Maltodextrin | 100:0 | 75:25 | 50:50 |

A tablet was prepared for each of the formulations. Each tablet was prepared by mixing the components listed hereinabove for each example in a suitable blender such as a V blender for about 1.5 to 2 hours and then compressing the mixture using a tablet press.

The dissolution profile of each tablet was carried out in a pH 5 acetate buffer using USP II apparatus at 50 rpm. The release profiles are depicted in Table 13.

TABLE 13
Release Profile of Clarithromycin Formulations

| | Cumulative % Released | | |
|---|---|---|---|
| | Formulation I | | |
| | (Comparative | Formulation II | Formulation III |
| Time (Hours) | Example 4) | Example 12 | Example 13 |
| 1 | 58 | 14 | 11 |
| 3 | 85 | 35 | 24 |
| 5 | 93 | 49 | 41 |
| 7 | 93 | 64 | 58 |
| 9 | | | 73 |
| 12 | | | 79 |

As used herein, the term “highly water soluble” means that the solubility of the material is at least about 1 gm/10 ml of H₂O at 25° C.

As used herein, the term “water soluble” means that the solubility of the material is at least about 1 gm/10 ml of H₂O at 25° C.

The term “water insoluble” is used in its normal sense. It is meant to imply that the solubility of the material in water at 25° C. is low, e.g., less than about 1 gm/ml of H₂O at 25° C.

The term “partially soluble” is meant that the solubility of the material at 25° C. lies between that of “water soluble” and that of “water insoluble”.

The above preferred embodiments and examples were given to illustrate the scope and spirit of the present invention. These embodiments and examples will make apparent to those skilled in the art other embodiments and examples. The other embodiments and examples are within the contemplation of the present invention. Therefore, the present invention should be limited only by the amended claims.

What is claimed is:

1. In a method for preparing an oral sustained release pharmaceutical composition in solid dosage form having a desired drug release profile, which pharmaceutical composition is prepared by mixing a drug in a therapeutically effective amount with an effective amount of a sustained release carrier to retard the release of the drug from the pharmaceutical composition and a water insoluble or partially water insoluble cellulose in an amount effective to enhance the ability of the pharmaceutical composition to form the solid dosage form, resulting in a pharmaceutical composition having a drug release profile exhibiting a faster release than that of the desired drug release profile, the improvement comprising adding to the pharmaceutical composition an effective amount of a maltodextrin to retard the rate of release of the drug in the sustained release pharmaceutical composition to the desired drug release profile when placed in aqueous system, the weight ratio of the maltodextrin to the water insoluble or partially water insoluble cellulose that is added to enhance tableting ranging from about 1:50 to about 50:1.

2. The improved method according to claim 1 wherein the water insoluble or partially water insoluble cellulose is starch or microcrystalline cellulose.

3. The improved method according to claim 1 wherein the cellulose is microcrystalline cellulose.

4. The improved method according to claim 3 wherein the cellulose is silicified microcrystalline cellulose.

5. The improved method according to claim 1 additionally containing additives.

6. The improved method according to claim 1 wherein the sustained release carrier is polymethylacrylate.

7. The improved method according to claim 1 wherein the sustained release carrier is a mixture of cellulose ether and xanthan gum in a weight ratio ranging from about 1:01 to about 1:2

8. The method according to claim 7 wherein the cellulose ether is hydropropylmethyl cellulose.

9. The improved method according to any one of claims 1-8, wherein the weight ratio of maltodextrin to cellulose ranges from about 1:20 to about 20:1.

10. The improved method according to claim 9, wherein the weight ratio of maltodextrin to cellulose ranges from 1:9 to about 9:1.

11. The improved method according to claim 1 wherein the sum of the maltodextrin and the cellulose ranges from about 5 to about 95% of the pharmaceutical composition.

12. In a method for preparing an oral sustained release pharmaceutical composition in tablet form having a desired drug release profile, which pharmaceutical composition is prepared by mixing a drug in a therapeutically effective amount, an effective amount of an sustained release carrier to retard the release of the drug from the pharmaceutical composition and a lubricating effective amount of a lubricant with a tableting effective amount of microcrystalline cellulose to enhance the ability of the pharmaceutical composition to form a tablet, resulting in a pharmaceutical composition having a drug release profile having a faster release than that of the desired drug release profile, the improvement comprising adding to the pharmaceutical composition an effective amount of maltodextrin to retard the rate of release of the drug in the sustained release pharmaceutical composition to the desired drug release profile when placed into an aqueous system, the weight ratio of the maltodextrin to the microcrystalline cellulose that is added to enhance the tableting ranging from about 1:50 to about 50:1.

13. The improved method according to claim 12 wherein the cellulose is silicified microcrystalline cellulose.

14. The improved method according to claim 12 additionally containing additives.

15. The improved method according to claim 12 wherein the sustained release carrier is polymethylacrylate.

16. The improved method according to claim 12 wherein the sustained release carrier is a mixture of cellulose ether and xanthan gum in a weight ratio ranging from about 1:01 to about 1:2

17. The method according to claim 16 wherein the cellulose ether is hydropropylmethyl cellulose.

18. The improved method according to claim 1 wherein the sustained release carrier is a mixture of cellulose ether and xanthan gum, such that the xanthan gum is present in the pharmaceutical formulation in an amount ranging from 3% to about 7% by weight of the tablet, said cellulose ether being present in an amount ranging from about 3% to about 20% by weight of the tablet, and the water insoluble cellulose is silicified microcrystalline cellulose, the weight ratio of maltodextrin to silicified microcrystalline cellulose ranging from about 1:20 to about 20:1.

19. The improved method according to claim 18 wherein the weight ratio of maltodextrin to silicified microcrystalline cellulose ranges form about 1:9 to about 9:1.

20. The improved method according to claim 18 or 19 wherein the cellulose ether is hydroxypropylmethyl cellulose.

21. The improved method according to claim 1 or 18 wherein the drug is metformin.

22. The improved method according to claim 1 or 18 wherein the drug is carbamazepine.

23. The improved method according to claim 1 or 18 wherein the drug is metroindazole, and the sustained release carrier is polymethacrylate.

24. The improved method according to claim 21 wherein the weight ratio of the maltodextrin to the microcrystalline cellulose ranges from about 1:9 to about 9:1.

25. The improved method according to claim 22 wherein the weight ratio of the maltodextrin to the microcrystalline cellulose ranges from about 1:9 to about 9:1.

26. The improved method according to claim 23 wherein the weight ratio of the maltodextrin to the microcrystalline cellulose ranges from about 1:9 to about 9:1.

27. A method of reducing the release profile of a drug in an aqueous medium in a controlled release pharmaceutical composition which pharmaceutical composition comprises a therapeutically effective amount of a medicament, a controlled release carrier and said method comprising adding thereto a partially water soluble or water insoluble cellulose in amounts sufficient to enhance the tableting ability of said pharmaceutical composition and maltodextrin in an amount sufficient to retard the release profile.

28. The method according to claim 27 wherein the weight ratio of said cellulose to maltodextrin ranges from amount 1:50 to about 50:1.

29. The method according to claim 27 wherein the water insoluble or partially soluble cellulose is starch or silicified microcrystalline cellulose.

30. The method according to claim 27 additionally containing adjuvants.

31. The method according to claim 27 wherein the sustained release carrier is polymethylacrylate.

32. The method according to claim 27 wherein the sustained release carrier is a mixture of a cellulose ether and xanthan gum in a weight ratio ranging from about 1:01 to about 1:2

33. The method according to claim 32 wherein the cellulose ether is hydropropylmethyl cellulose.

34. The improved method according to claim 28, wherein the weight ratio of the water insoluble or partially soluble cellulose to maltodextrin ranges from about 1:20 to about 20:1.

35. The method according to claims 28, wherein the weight ratio of water insoluble or partially soluble cellulose to maltodextrin ranges from about 1:9 to about 9:1.

36. The method according to claim 27 wherein the sum of the maltodextrin and the cellulose ether ranges from about 5 to about 90% of the pharmaceutical composition.

37. The method according to any one of claims 1, 12 and 27 wherein the sustained release carrier is a hydrophilic polymer, hydrophobic polymer or wax polymer.

38. A sustained release pharmaceutical composition in oral dosage form comprising a pharmaceutically effective amount of a drug, a sustained release carrier in an effective amount to retard the release of the drug from said composition when placed in an aqueous system, a lubricating effective amount of a lubricant, a water insoluble or partially water insoluble cellulose and maltodextrin, wherein the weight ratio of cellulose to maltodextrin ranges from about 50:1 to 1:50.

39. The pharmaceutical composition according to claim 38 wherein the sustained release polymer is polymethacrylate.

40. The pharmaceutical composition according to claim 38 wherein the sustained release polymer is a mixture of cellulose either and xanthan gum.

41. The pharmaceutical composition according to claim 40 wherein the weight ratio of cellulose ether to xanthan gum ranges from about 1:0.1 to about 1:2.

42. The pharmaceutical composition according to claim 40 wherein the cellulose ether is hydroxypropylmethyl cellulose.

43. The pharmaceutical composition according to claim 38 wherein the weight ratio of silicified microcrystalline cellulose to maltodextrin ranges from about 20:1 to about 1:20.

44. The pharmaceutical composition according to claim 43 wherein the weight ratio of cellulose to maltodextrin ranges from about 9:1 to about 1:9.

45. The pharmaceutical composition according to claim 38 wherein the drug is metformin, metronidazole or carbamazepine or mesalamine.

46. The pharmaceutical composition according to claim 38 wherein the water insoluble or partially water insoluble cellulose is starch or microcrystalline cellulose.

47. The pharmaceutical composition according to claim 46 wherein the water insoluble or partially water insoluble cellulose is microcrystalline cellulose.

48. The pharmaceutical composition according to claim 47 wherein the microcrystalline cellulose is silicified microcrystalline cellulose.

49. A method of treating a disease in a patient requiring a sustained release formulation of a drug for treating said disease, said treatment comprising administering to the patient a pharmaceutically effective amount of the sustained release pharmaceutical composition according to any one of claims 38-48.

50. The improved method according to claim 1 wherein the sustained release carrier is glyceryl behenate.

51. The improved method according to claim 12 wherein the sustained release carrier is glyceryl behenate.

52. The method according to claim 27 wherein the sustained release carrier is glyceryl behenate.

53. The sustained release pharmaceutical composition according to claim 38 wherein the sustained release carrier is glyceryl behenate.

54. The sustained release pharmaceutical composition according to claim 38 wherein the sum of the maltodextrin and the cellulose ranges from about 5% to about 95% by weight of the pharmaceutical composition.

55. The sustained release pharmaceutical composition according to claim 54 wherein the sum of the maltodextrin and the cellulose ranges from about 10% to about 60% by weight of the pharmaceutical composition.

56. The sustained release pharmaceutical composition according to claim 55 whereon the sum of the maltodextrin and the cellulose ranges from about 20% to about 50% by weight of the pharmaceutical composition.

57. The improved method according to claim 1 wherein the solid dosage oral form is a pellet, tablet or capsule.

58. The method according to claim 27 wherein the solid dosage oral form is a pellet, tablet or capsule.

59. The pharmaceutical composition according to claim 38 wherein the solid unit dosage oral form is a pellet, tablet or capsule.

60. The pharmaceutical composition according to the claim 38 wherein the sustained release carrier is a hydrophilic polymer.

61. The pharmaceutical composition according to claim 38 wherein the sustained release carrier is a hydrophobic polymer.

62. The pharmaceutical composition according to claim 38 wherein the sustained release carrier is a wax polymer.
